SEQUENCE LISTING (5 sequences)

SEQ ID NO: 1 - protein, 43 aa, <Unknown>
  hypothetical: <Unknown>
  anti-sense: <Unknown>
  original source individual isolate: <Unknown>
  original source cell type: <Unknown>
  immediate source library: <Unknown>
  immediate source clone: <Unknown>
  relevant residues in seq id no: 1:FROM 1 TO 43
DAEFRHDSGYEVHHQKLVFFAEDVGSNKGAIIGLMVGGVVIAT

SEQ ID NO: 2 - protein, 42 aa [sequence omitted]

SEQ ID NO: 3 - protein, 41 aa
  Signal Sequence 1-41: IDENTIFICATION METHOD: Similarity to other sequences, hydro-phobic
  relevant residues in seq id no: 3:FROM 1-41
DAEFRHDSGYEVHHQKLVFFAEDVGSNKGAIIGLMVGGVVI

SEQ ID NO: 4 - protein, 40 aa
  Signal Sequence 1-40: IDENTIFICATION METHOD: Similarity to other sequences, hydro-phobic
  relevant residues in seq id no: 4:FROM 1-40
DAEFRHDSGYEVHHQKLVFFAEDVGSNKGAIIGLMVGGVV

SEQ ID NO: 5 - protein, 39 aa
  Signal Sequence 1-39: IDENTIFICATION METHOD: Similarity to other sequences, hydro-phobic
  relevant residues in seq id no: 5:FROM 1-39
DAEFRHDSGYEVHHQKLVFFAEDVGSNKGAIIGLMVGGV

(19) United States
(12) Patent Application Publication
(10) Pub. No.: US 2002/0182660 A1
(43) Pub. Date: Dec. 5, 2002

(54) N- and C-terminus specific immunoassays for full length beta-amyloid peptide-Abeta(1-40), Abeta(1-39), Abeta(1-40), Abeta(1-42) , and Abeta(1-43)

(76) Inventor: Kei-Lai L. Fong, King of Prussia, PA (US)

Correspondence Address: KAO H. LU, 686 LAWSON AVE, HAVERTOWN, PA, 19083, US

(21) Appl. No.: 10/051,496

(22) Filed: Jan. 18, 2002

Related U.S. Application Data
(62) Division of application No. 09/784,854, filed on Feb. 16, 2001.
(60) Provisional application No. 60/183,407, filed on Feb. 18, 2000.

(51) Int. Cl. G01N 33/53; G01N 33/537; G01N 33/543

(52) U.S. Cl. 435/7.93

(57) ABSTRACT

This invention describes a specific, sensitive, reproducible and multiplexed assay for simultaneous quantification of full-length beta-amyloid peptides in biological matrices; a method to monitor and measure the hallmarks associated with the progress of Alzheimer's disease. This invention employs one specific antibody that recognizes the N-terminus of all the abeta peptides, and a panel of detection antibodies that distinguish each abeta peptide by their sequence difference at the C-terminus. Each of these C-terminus specific antibodies carries a different label (or tag), for example, fluorescence labels with different excitation/emission characteristics or electrochemiluminescence (ECL) label with different excitation/emission characteristics. This invention allows simultaneous quantification of several abeta peptides in one single assay. The concept of using N- and C-terminus specific antibodies to capture the each of the said abeta peptides by both ends is the basis of providing the desired specificity.

### CROSS REFERENCE APPLICATIONS

This application is a divisional application form the patent application Ser. No. 09/784,584 filed on Feb. 16, 2001, which claims priority from Provisional Patent Application No. 60/183,407, filed on Feb. 18, 2000.

### BACKGROUND

This invention relates to detection of Alzheimer's disease, more particularly, an assay method and apparatus, which can monitor and measure the hallmarks associated with the progress of Alzheimer's disease.

One of the major pathological hallmarks of the neuropathology of Alzheimer's disease is the progressive deposition of fibrillar beta-amyloid peptide (Abeta) into neuritic and diffuse plagues in the brain parenchyma. The physiological function/regulation of Abeta/APP is not fully understood. However, it is very likely that one or more of these Abeta variants is an important biomarker for the development of Alzheimer's disease. The levels of different amyloid peptide variants in the same biological compartment (or sample) provide key information in understanding the pathogenesis of Alzheimer's disease. The ability to measure the deposed different beta-amyloid peptide variants quantitatively from a target sample is a useful tool to detect and monitor the progress of Alzheimer's disease.

Beta-amyloid peptide has a heterogeneous COOH terminus, as variants with 39 to 43 amino acid residues. These Abeta peptides are proteolytic products derived from the amyloid precursor protein. The Abeta(1-39), FIG. 1e, [SEQ ID:5], and Abeta(1-40), FIG. 1d,[SEQ ID:4], peptide were reported to be the dominant forms associated with the blood vessel amyloid whereas Abeta(1-42), FIG. 1b, [SEQ ID:2], and Abeta (1-43), FIG. 1a, [SEQ NO:1], were the dominant forms found in amyloid neuritic plague.

### SUMMARY

A method for detecting the level of specific full length Abeta amyloid peptide levels in a sample is disclosed. A first antibody is introduced that binds and capture multiple types of Abeta peptide, such as Abeta peptides (1-39), [SEQ IS:5], FIG. 1e, (1-40), [SEQ ID:4] FIG. 1d, (1-41) [SEQ ID:3], FIG. 1c, (1-42) [SEQ ID:2], FIG. 1b, and(1-43) [SEQ ID:1] FIG. 1a. A second antibody is introduced that binds and captures a specific one of the Abeta peptides at a different location from the first antibody. The types of antibodies used can be either monoclonal or polyclonal or the combination of both. The second antibody provides an identifiable tage so that the level of Abeta peptides bound with both said first and second antibodies can be measured. Preferably the first antibody binds with the N-terminus of the Abeta peptide and the second antibody binds with the C-terminus of the Abeta peptide, so that only full length Abeta peptides are tagged and measured. The process enables measurement of individual Abeta peptide levels useful for tracking the progression of Alzheimer' disease.

The method providing more specific and sensitive bio-markers to monitor the progress of Alzheimer's Disease including steps of:

Tagging a plurality of specified antibodies with a plurality of labeling techniques;

Identifying each of specific peptides associated with said disease by binding said tagged antibodies at specific regions of said peptide; and

Measuring the result of binding to determine the threshold of said disease.

### BRIEF DESCRIPTION OF THE DRAWINGS

FIGS. 1a-1e show amino acid structures for Abeta amyloid peptide (1-39), (1-40), (1-41), (1-42) and (1-43).

FIG. 2 shows a diagram of a multiplexed immunoassay for Abeta amyloid peptide.

FIG. 3 shows a result of the N and C-terminus specific immunoassay for Abeta amyloid peptide (1-42).

FIG. 4 Shows another result of the N and C-terminus specific immunoassay for Abeta amyloid (1-42).

FIG. 5 shows a result of the N and C-terminus specific immunoassay for Abeta amyloid (1-42) in human plasma.

FIG. 6 shows the result of the N and C-terminus specific immunoassay for Abeta amyloid (1-40).

### DETAILED DESCRIPTION OF THE PREFERRED EMBODIMENTS

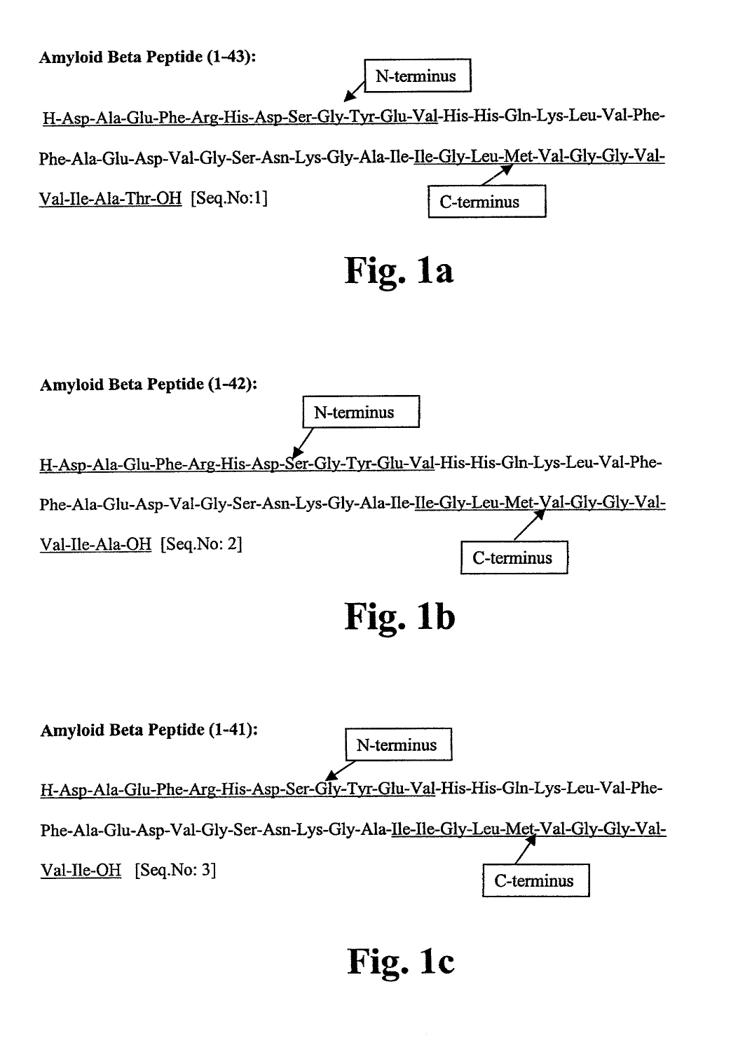

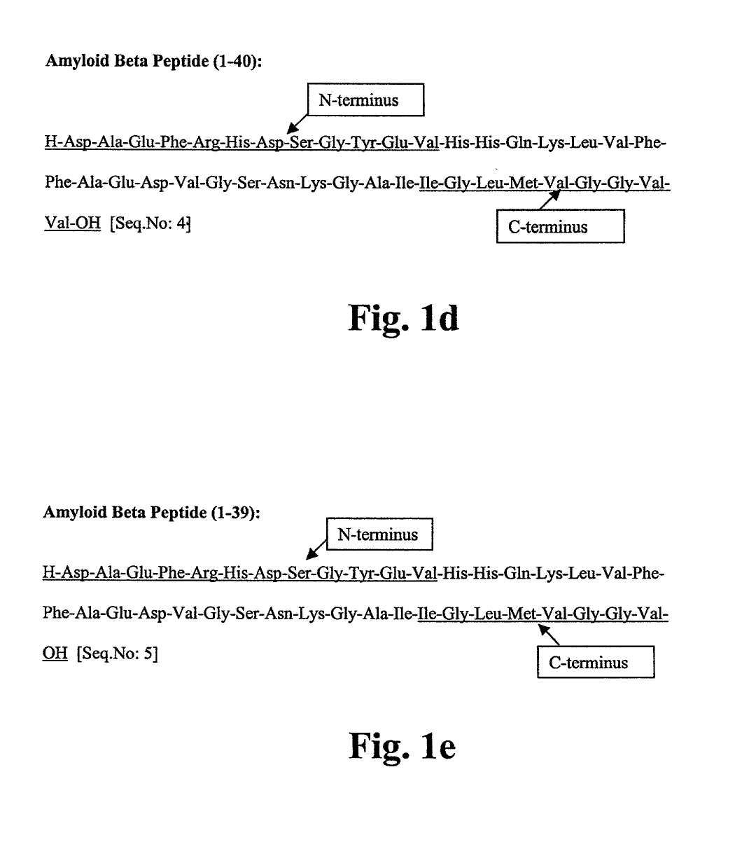

FIG. 1 shows the two dimensional amino acid chain structure of Abeta amyloid peptide (1-42), FIG. 1b, where the left side area is called N-terminus and the right side area is called C-terminus. Abeta peptide variants (1-39), FIG. 1e, (1-40), FIG. 1d, (1-41), FIG. 1c, and (1-43), FIG. 1a, have the similar amino acid chain structure as (1-42), FIG. 1b, has, but have different number of amino acid in their prospective chain. Particular antibodies have been identified specifically for either the N, or C-terminus of each of Abeta peptide variants. For example, one is the mouse monoclonal antibody, such as clone number BAM-10 that recognizes the amino acids 1-12 (N-terminus) of an Abeta peptide, and an affinity purified antibody that recognizes the C-terminus of the Abeta (1-42).

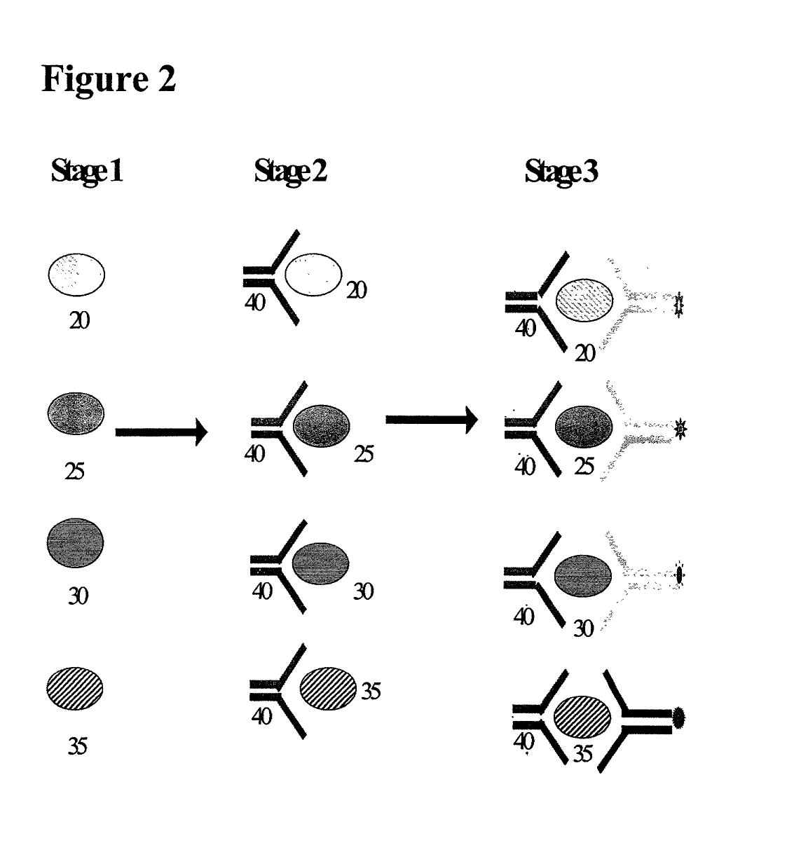

FIG. 2 illustrates a three-staged multiplexed immuno assay for beta amyloid peptides.

In Stage 1 of FIG. 2, Abeta(1-39), Abeta(1-40), Abeta(1-42) and Abeta(1-43), 20,25,30 and 35, respectively, are in microsphere. In stage 2 of FIG. 2, the N-terminus specific antibody, 40, will bind and capture Abeta peptides(1-39), (1-40), (1-42) and Abeta (1-42), 20,25,30 and 35, respectively. In stage 3, different C-terminus specific antibodies, 45, 50, 55 and 60, each labeled with different fluorescence tags or chemiluminesence tags, are utilized 45-60 as another screen tool, whereby various Abeta peptides can be further characterized and distinguished. As long as the binding of an N-terminus specific antibody to the Abeta peptide does not exclude the binding of a C-terminus specific antibody to the Abeta peptide, use of both kinds of antibody can be a powerful screen tool to identify and quantize Abeta peptide having both C- and N-terminus.

This invention only detects intact, full-length Abeta peptide (and/or its COOH terminal variants), but not its biologically distinguishable fragments or its aggregates (fibrils, or any polymeric forms).

Several experimental results support the invention. For example, in a sample mixture of Abeta peptide (1-40) or (1-42) which is first run through an ELISA plate which is coated with the n-terminus specific antibody, both Abeta peptides (1-40) and (1-42) are captured by the N-terminus specific antibody. Next a C-terminus specific antibody, such as affinity purified rabbit polyclonal anti-Abeta(1-40): PharMingen lot number M029157, labeled with fluorescence (pretreated excitation at 485 nm frequency emission with 538 nm frequency) is applied to the captured Abeta peptides. The C-terminus antibody reacts with fluorescence—Ex succinimidyl estex then using any exhaust dislysis to remove excess fluorescence—Ex succinimidyl ester.

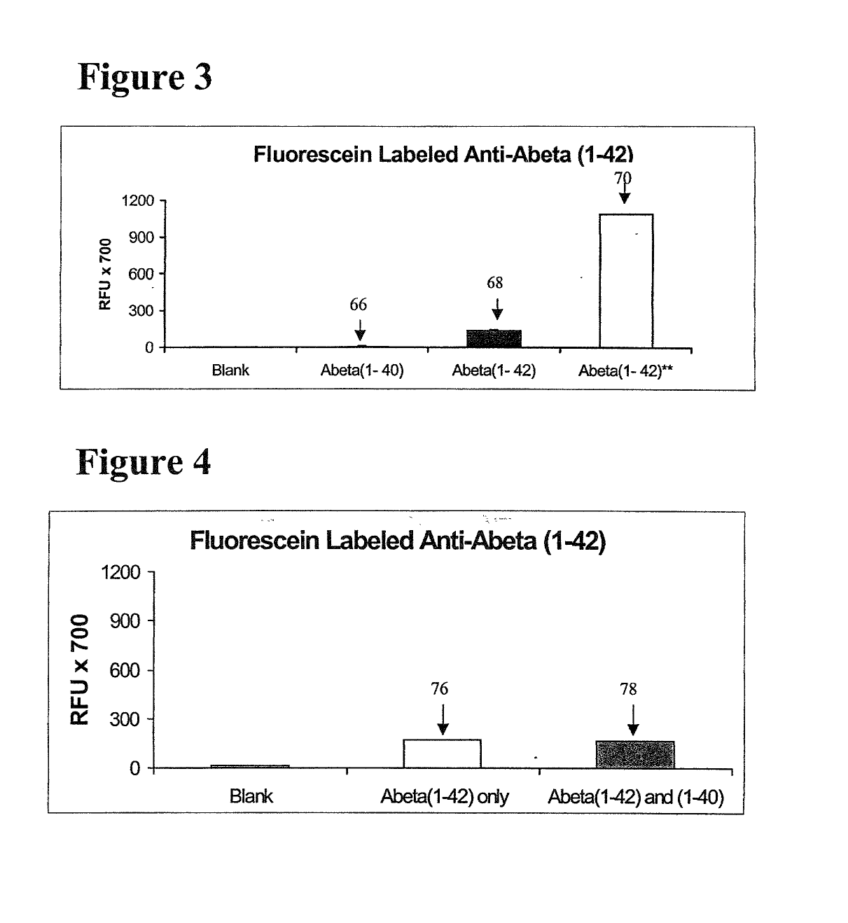

FIG. 3 shows the result of this process. The x axis indicates the intensity of fluorescence which in turn directly correlating to the concentration of N-terminus specific antibody captured Abeta peptide bound with the fluorescence labeled C-terminus specific antibody. The data shows the concentration of captured Abeta(1-40), histogram 66, bound with the particular fluorescence labeled C-terminus antibodies, such as affinity purified rabbit polyclonal anti-Abeta(1-42): PharMingen lot number M050781 is almost close to undetectable level. This process does capture Abeta(1-42). The result is supported by the histogram 68 shown in FIG. 3. If the sequence of applying N- and C-terminus specific antibodies is reversed, by first, applying fluorescence labeled C-terminus antibody to the (1-40) and (1-42) mixture sample and next running the resulting complex through the N-terminus specific antibody coated ELISA plate, this C- first then N- next sequence of applying antibodies significantly increases the amount of captured Abeta(1-42) as much as six (6) fold comparing to the N- first the C- next sequence. This indicates that the order of applying different C- and N-specific antibodies to the sample has different effects. The result is supported by histogram 70.

In another experiment, shown in FIG. 4, the above process is applied to two type samples, one with Abeta(1-42), only, and another with the same concentration of Abeta(1-42) plush Abeta(1-40). Referring to FIG. 4, the histogram 76 shows the Abeta(1-42) only sample has the same fluorescence intensity level as the sample of mixture of Abeta! 1-42) and (1-40) in histogram 78. The logical explanation for the results of FIG. 4 is that this invention will reliably measure the concentration of Abeta(1-42) in the sample with or without the presence of other Abeta peptides.

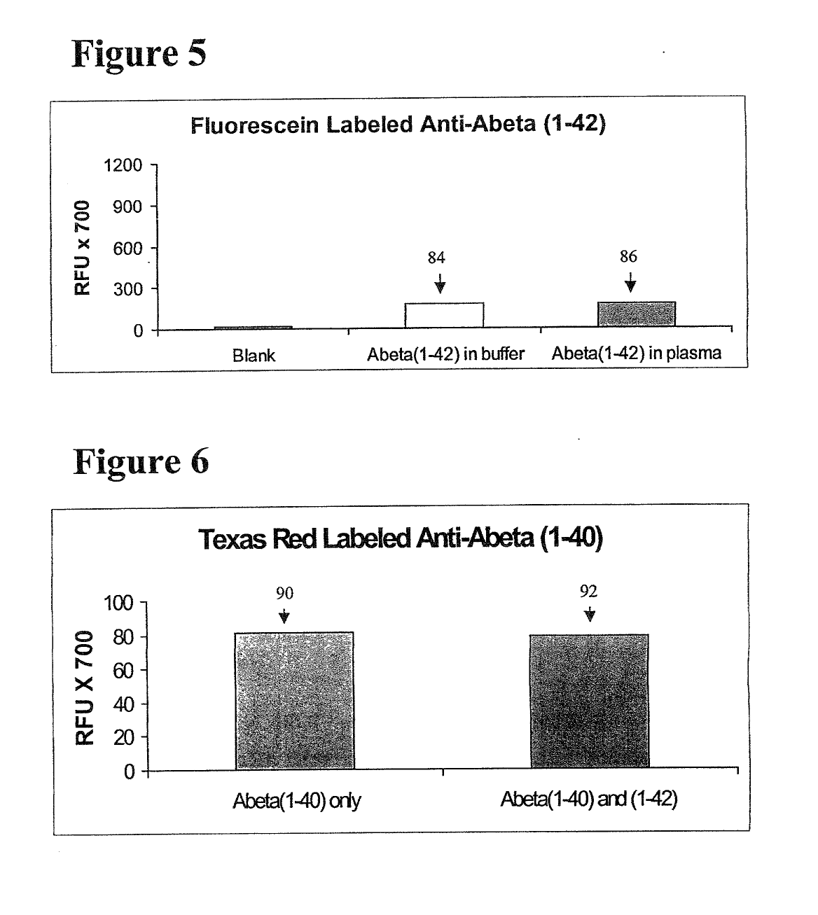

The invention further demonstrates that the above process works on sample extracted from human tissue. FIG. 5 shows that the Abeta(1-42) sample in buffer, histogram 84, has the same fluorescence intensity level as the Abeta(1-42) sample in human plasma, histogram 86. The data shown in FIG. 5, supports that the invention can reliably measure the concentration of Abeta(1-42) from a human plasma.

Working with the same N-terminus specific antibody, the invention identifies another C-terminus specific antibody, such as affinity purified rabbit polyclonal anti-Abeta(1-40): PharMingen lot number M029157, labeled with Texas red (excitation 584, emission 612) for Abeta(1-40). The Texas Red labeled antibody was prepared by reacting the anti Abeta(1-40) antibody with Texas Red-X succinimidyl ester, excess Texas Red-x succinimidyl ester was removed by exhausted dialysis. A sample containing Abeta peptides was run through a N-terminus specific antibody coated ELISA plate. Abeta(1-40) and Abeta(1-42) peptides are captured by binding with the N-terminus specific antibody. The applying of C-terminus specific (1-40) antibody will bind with Abeta(l-40) only even with the Abeta(1-42) present. FIG. 6 shows that the Abeta(1-40) sample histogram 90, has the same fluorescence intensity level as the sample mixture of Abeta(1-40) and (1-42), histogram 92. Therefore, the data in FIG. 6 illustrates the reliable measurement of the concentration Abeta(1-40) only from a sample of mixture of (1-40) and (1-42) of Abeta peptides.

This invention presents a method and apparatus which uses different C-terminus specific antibodies labeled with different fluorescent tags or chemiluminescence tags in combination with N-terminus specific antibody which can reliably measure concentration of each Abeta peptide variants from human tissue.

What is claimed is:

1. A method of enabling measurement of the full length Abeta peptide level of a specific Abeta peptide in a sample containing multiple types of Abeta peptide comprising;
Capturing and binding one terminus of the multiple types of Abeta peptides with a first antibody;
Capturing and binding the specific Abeta peptide at an opposite non overlapping terminus with a second peptide such that said second antibody provides a tag to enable measurement of the full length Abeta peptides captured and bound by both first and second antibodies and such that said second antibody does not bind with any other of the multiple types of Abeta peptide.

2. A method according to claim 1 wherein the first antibody binds with an N-terminus of the Abeta peptides and the second antibody binds with a C-terminus of the specific type of Abeta peptides.

3. A method according to claim 2 wherein the multiple types of Abeta peptides consist of (1-39), (1-40), (1-42) and (1-43) and the specific Abeta peptide is (1-42) and wherein:
A N-terminus specific antibody is used as said first antibody; and
A C-terminus specific antibody is used as said second antibody.

4. The method to claim 3 wherein the N-terminus specific antibody is the anti-Abeta mouse monoclonal antibody.

5. The method to claim 3 wherein the C-terminus specific antibody is an affinity purified rabbit polyclonal anti-Abeta(1-40): PharMingen lot number M029157 for peptide (1-42) and an affinity purified rabbit polyclonal anti-Abeta(1-42); PharMingen lot number M050781 for peptide (1-42).
